(19) United States
(12) Patent Application Publication
(10) Pub. No.: US 2019/0177265 A1
(43) Pub. Date: Jun. 13, 2019

(54) METHOD FOR COUPLING A FIRST COMPOUND TO A SECOND COMPOUND

(71) Applicant: Dow AgroSciences LLC, Indianapolis, IN (US)

(72) Inventors: Patrick S. Hanley, Midland, MI (US); Thomas P. Clark, Midland, MI (US); Matthias S. Ober, Midland, MI (US); William J. Kruper, Sanford, MI (US)

(73) Assignee: Dow AgroSciences LLC, Indianapolis, IN (US)

(21) Appl. No.: 16/271,008

(22) Filed: Feb. 8, 2019

Related U.S. Application Data
(63) Continuation of application No. 15/515,071, filed on Mar. 28, 2017, now abandoned.
(60) Provisional application No. 62/149,237, filed on Apr. 17, 2015.
(60) Provisional application No. 62/061,641, filed on Oct. 8, 2014.

(51) Int. Cl. C07C 209/18; C07D 317/66; C07C 209/66; B01J 31/22; C07D 295/023; C07C 213/02; C07B 43/04; C07D 295/033; C07D 317/58; C07D 213/74; C07C 253/30; C07C 231/12; C07C 227/08; C07C 221/00

(52) U.S. Cl. CPC C07C 209/18; B01J 2231/44; C07C 209/66; B01J 31/2295; C07D 295/023; C07C 213/02; C07B 43/04; C07D 295/033; C07D 317/58; C07D 213/74; C07C 253/30; C07C 231/12; C07C 227/08; C07C 221/00; B01J 2531/824; C07D 317/66

(57) ABSTRACT

The present disclosure describes a method of coupling a first compound to a second compound, the method comprising: providing the first compound having a fluorosulfonate substituent; providing the second compound comprising an amine; and reacting the first compound and the second compound in a reaction mixture, the reaction mixture including a catalyst having at least one group 10 atom, the reaction mixture under conditions effective to couple the first compound to the second compound. The present disclosure further describes a one-pot method for coupling a first compound to a second compound.

### CROSS REFERENCE TO RELATED APPLICATIONS

This application is a continuation of, and claims the benefit of, application Ser. No. 15/515,071, filed on Mar. 28, 2017, and which is the National Stage Entry of International Application PCT/US15/54653, filed Oct. 8, 2015, and which claims the benefit of, and priority from, U.S. Provisional Patent Application Ser. No. 62/061,641 filed Oct. 8, 2014 and U.S. Provisional Patent Application Ser. No. 62/149,237 filed Apr. 17, 2015. The entire contents of all of the above-identified applications are hereby incorporated by reference into this application.

### BACKGROUND

Buchwald-Hartwig coupling (C—N coupling) is a valuable synthetic method for coupling compounds, thereby forming a new carbon-nitrogen bond between a first compound and a second compound. Traditionally, C—N coupling partners consist of a first compound having a halide or sulfonate substituent and a second compound comprising an amine. It is common for the first compound to comprise an aryl compound.

It is known that triflates (trifluoromethanesulfonate), having the formula F₃CSO₂—, may be used in the place of the halides in C—N couplings, however the expense of triflic anhydride (CF₃SO₂)₂O has limited the use of triflates in C—N couplings to the production of fine chemicals. Further, the atom economy of triflic anhydride is low since half of the molecule is expended as monomeric triflate anion (CF₃SO₂⁻) as a result of condensation with a phenolic precursor.

It is also known that aryl methanesulfonates (also known as mesylates) are suitable for aryl-amine cross-coupling reactions. One drawback of aryl-amine crosscouping using aryl methanesulfonates is that these reactions require expensive palladium catalysts. Another drawback of aryl-amine cross-coupling reactions using aryl methanesulfonates is low atom economy.

When performing a C—N coupling using either a triflate or methanesulfonate, it is common to perform the reaction in two steps, a first step comprising replacing the hydroxyl group on the first aromatic compound with the triflate or the methanesulfonate, and a second step comprising coupling the first compound with the second compound. A separation step is generally required between the first and second steps.

It would be desirable to have a replacement for triflates and methanesulfonates which allow the C—N coupling reactions. Furthermore, a water-stable triflate replacement could ensure lower loadings of expensive catalysts. In addition, it is desired to have a cross-coupling reaction which has greater atom economy.

### STATEMENT OF INVENTION

The present disclosure describes a method of coupling a first compound to a second compound, the method comprising: providing the first compound having a fluorosulfonate substituent; providing the second compound comprising an amine; and reacting the first compound and the second compound in a reaction mixture, the reaction mixture including a catalyst having at least one group 10 atom, the reaction mixture under conditions effective to couple the first compound to the second compound.

The present disclosure describes a method for coupling a first compound A¹ a second compound A², as illustrated in Equation 1, comprising:

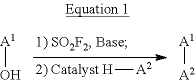

providing the first compound A¹ having a hydroxyl substituent, sulfuryl fluoride and a base to a reaction mixture, the first compound A¹ comprising an aryl group or a heteroaryl group;

providing a catalyst comprising a group 10 atom and the second compound A² to the reaction mixture, the second compound A² as defined in Equation 4:

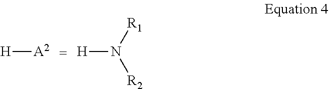

the second compound A² comprising an amine wherein each of R¹ and R² are independently Hydrogen, an aryl group, a heteroaryl group, an alkyl group, a cycloalkyl group, a nitro group, a halide, a nitrogen, a cyano group, a carboxyester group, an acetoxy group, a substituted alkyl, aryl, heteroaryl or cycloalkyl group, or R¹ and R² are constituent parts of a ring system;

reacting the first compound and the second compound under conditions effective to couple the first compound to the second compound.

### DETAILED DESCRIPTION

Unless otherwise indicated, numeric ranges, for instance “from 2 to 10,” are inclusive of the numbers defining the range (e.g., 2 and 10).

Unless otherwise indicated, ratios, percentages, parts, and the like are by moles.

As used herein, unless otherwise indicated, the phrase “molecular weight” refers to the number average molecular weight as measured in the conventional manner.

“Alkyl,” as used in this specification, whether alone or as part of another group (e.g., in dialkylamino), encompasses straight and branched chain aliphatic groups having the indicated number of carbon atoms. If no number is indicated (e.g., aryl-alkyl-), then 1-12 alkyl carbons are contemplated. Preferred alkyl groups include, without limitation, methyl, ethyl, propyl, isopropyl, butyl, isobutyl, sec-butyl, tert-butyl, pentyl, hexyl and tert-octyl.

The term “heteroalkyl” refers to an alkyl group as defined above with one or more heteroatoms (nitrogen, oxygen, sulfur, phosphorus) replacing one or more carbon atoms within the radical, for example, an ether or a thioether.

An “aryl” group refers to any functional group or substituent derived from an aromatic ring. In one instance, aryl refers to an aromatic moiety comprising one or more aromatic rings. In one instance, the aryl group is a C₆-C₁₈ aryl group. In one instance, the aryl group is a C₆-C₁₀ aryl group. In one instance, the aryl group is a C₁₀-C₁₈ aryl group. Aryl groups contain 4n+2 pi electrons, where n is an integer. The aryl ring may be fused or otherwise attached to one or more heteroaryl rings, aromatic or non-aromatic hydrocarbon rings or heterocycloalkyl rings. Preferred aryls include, without limitation, phenyl, naphthyl, anthracenyl, and fluorenyl. Unless otherwise indicated, the aryl group is optionally substituted with 1 or more substituents that are compatible with the syntheses described herein. Such substituents include, but are not limited to, sulfonate groups, boron-containing groups, alkyl groups, nitro groups, halogens, cyano groups, carboxylic acids, esters, amides, C₂-C₈ alkene, and other aromatic groups. Other substituents are known in the art. Unless otherwise indicated, the foregoing substituent groups are not themselves further substituted.

“Heteroaryl” refers to any functional group or substituent derived from an aromatic ring and containing at least one heteroatom selected from nitrogen, oxygen, and sulfur. Preferably, the heteroaryl group is a five or six-membered ring. The heteroaryl ring may be fused or otherwise attached to one or more heteroaryl rings, aromatic or non-aromatic hydrocarbon rings or heterocycloalkyl rings. Examples of heteroaryl groups include, without limitation, pyridine, pyrimidine, pyridazine, pyrrole, triazine, imidazole, triazole, furan, thiophene, oxazole, thiazole. The heteroaryl group may be optionally substituted with one or more substituents that are compatible with the syntheses described herein. Such substituents include, but are not limited to, fluorosulfonate groups, boron-containing groups, C₁-C₈ alkyl groups, nitro groups, halogens, cyano groups, carboxylic acids, esters, amides, C₂-C₈ alkene and other aromatic groups. Other substituents are known in the art. Unless otherwise indicated, the foregoing substituent groups are not themselves further substituted.

“Aromatic compound” refers to a ring system having 4n+2 pi electrons where n is an integer.

As noted above, the present disclosure describes a process for coupling a first compound to a second compound. This process is shown generally in Equation 1, whereby a first compound having a hydroxyl group is first reacted with SO₂F₂ and a base and is second reacted with a second compound comprising an amine in the presence of a catalyst. It is understood that where a hydroxyl group is indicated, the hydroxyl group could be deprotonated to form a phenolate (e.g. the deprotonation step could be performed prior to introduction of A¹ to the reaction mixture or after the introduction to the reaction mixture).

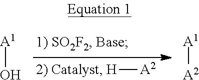

Unexpectedly, it has been found that the reaction of Equation 1 may be performed as a one-pot reaction, as compared to performing the reaction in discrete steps. Without being limited by theory, it is anticipated that the reaction shown in Equation 1 proceeds along the same reaction path whether performed as a one-pot reaction or as discrete steps. When performed in discrete steps, the first step comprises reacting a first compound having a hydroxyl substituent with SO₂F₂ to yield the product shown in Equation 2, and the second step comprises reacting the product of Equation 2 with a second compound comprising an amine to yield the product shown in Equation 3.

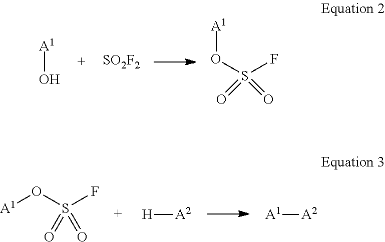

In one instance, the process involves a one-pot reaction where a first compound having a hydroxyl group is first reacted with SO₂F₂ and a base and is second reacted with a second compound comprising an amine in the presence of a catalyst, as shown generally in Equation 1. Without being limited by theory, it is expected that Equation 3 is the same general reaction as depicted by step 2) of the reaction shown in Equation 1.

As used in Equation 1, Equation 2 and Equation 3, the first compound is identified as A¹. The first compound is either an aryl group or a heteroaryl group. The second compound is identified as A² as illustrated in Equation 4:

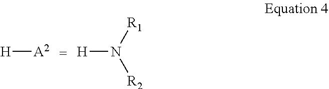

The second compound A² is an amine wherein R¹ and R² are each independently H or other suitable substituent suitable for use in a C—N coupling. In one instance, R¹ and R² are each independently H, alkyl or aryl groups. The result of the reactions shown in Equation 1 and Equation 3 is the formation of a new carbon-nitrogen bond between the first compound and the second compound, thereby coupling the first compound to the second compound.

As noted above in the first step of Equation 1 and in Equation 2, the first compound is bonded to a fluorosulfonate group. A fluorosulfonate group refers to O-fluorosulfonate of the formula —OSO₂F. O-fluorosulfonate may be synthesized from sulfuryl fluoride. The fluorosulfonate group serves as a leaving group from the first compound. Without being limited by theory, the sulfuryl atom of the fluorosulfonate group is bonded to the oxygen of the hydroxyl group of the first compound.

As noted above, the second compound is an amine. The amine is alternatively ammonia, a primary or a secondary amine. R¹ and R² are each independently a substituent suitable for use in a C—N coupling, for example, Hydrogen, aryl, heteroaryl, alkyl, heteroakyl, amide, carbonaryl, carbonheteroaryl, halide, Nitrogen, carbonyl or acetoxy. In one instance, the amine includes an R¹ and R² that are members of one or more rings, for example, a cyclic amine, a di-alkyl amine or di-aryl amine. In one instance, R¹ and R² are bonded to each other. In one instance, each of R¹ and R² are independently C₁₋₁₈ alkyl, C₃₋₁₈ cycloalkyl, C₆₋₁₈ aryl, or H. In one instance, the alkyl or aryl groups of the amine are themselves further substituted.

As noted above in Equation 1 and Equation 3, the first compound is reacted with the second compound in a reaction mixture. The reaction mixture includes a catalyst having at least one group 10 atom. In some instances, the reaction mixture also includes a ligand, and a base. The group 10 atoms include nickel, palladium and platinum.

The catalyst is provided in a form suitable to the reaction conditions. In one instance, the catalyst is provided on a substrate. In one instance, the catalyst having at least one group 10 atom is generated in situ from one or more precatalysts and one or more ligands. Examples of palladium precatalysts include, but are not limited to, Palladium(II) acetate, Palladium(II) chloride, Dichlorobis(acetonitrile)palladium(II), Dichlorobis(benzonitrile)palladium(II), Allylpalladium chloride dimer, Palladium(II) acetylacetonate, Palladium(II) bromideBis(dibenzylideneacetone)palladium(0), Bis(2-methylallyl)palladium chloride dimer, Crotylpalladium chloride dimer, Dichloro(1,5-cyclooctadiene)palladium(II), Dichloro(norbornadiene)palladium(II), Palladium(II) trifluoroacetate, Palladium(II) benzoate, Palladium(II) trimethylacetate, Palladium(II) oxide, Palladium(II) cyanide, Tris(dibenzylideneacetone)dipalladium(0), Palladium(II) hexafluoroacetylacetonate, cis-Dichloro(N,N,N′,N′-tetramethylethylenediamine) palladium(II), and Cyclopentadienyl[(1,2,3-n)-1-phenyl-2-propenyl]palladium(II).

In one instance, nickel-based catalysts are used. In another instance, platinum-based catalysts are used. In yet another instance, a catalyst including one or more of nickel, platinum and palladium-based catalysts are used.

In one instance, pyridine-enhanced precatalyst preparation stabilization and initiation (PEPPSI) type catalysts are used, for example, [1,3-Bis(2,6-Diisopropylphenyl)imidazol-2-ylidene](3-chloropyridyl)palladium(II) dichloride, and (1,3-Bis(2,6-diisopropylphenyl)imidazolidene) (3-chloropyridyl) palladium(II) dichloride.

Examples of nickel precatalysts include, but are not limited to, nickel(II) acetate, nickel(II) chloride, Bis(triphenylphosphine)nickel(II) dichloride, Bis(tricyclohexylphosphine)nickel(II) dichloride, [1,1′-Bis(diphenylphosphino)ferrocene]dichloronickel(II), Dichloro[1,2-bis(diethylphosphino)ethane]nickel(II), Chloro(1-naphthyl)bis(triphenylphosphine)nickel(II), 1,3-Bis(2,6-diisopropylphenyl)imidazolium chloride, Bis(1,5-cyclooctadiene)nickel(0), Nickel(II) chloride ethylene glycol dimethyl ether complex, [1,3-Bis(diphenylphosphino)propane]dichloronickel(II), [1,2-Bis(diphenylphosphino)ethane]dichloronickel(II), and Bis(tricyclohexylphosphine)nickel(0).

The ligand used in the reaction mixture is preferably selected to generate the selected catalyst from a pre-catalyst. For example, the ligand may be a phosphine ligand, a carbene ligand, an amine-based ligand, a carboxylate based ligand, an aminodextran, an aminophosphine-based ligands or an N-heterocyclic carbene-based ligand. In one instance, the ligand is monodentate. In one instance, the ligand is bidentate. In one instance, the ligand is polydentate.

Suitable phosphine ligands may include, but are not limited to, mono- and bi-dentate phosphines containing functionalized aryl or alkyl substituents or their salts. For example, suitable phosphine ligands include, but are not limited to, triphenylphosphine; Tri(o-tolyl)phosphine; Tris(4-methoxyphenyl)phosphine; Tris(pentafluorophenyl)phosphine; Tri(p-tolyl)phosphine; Tri(2-furyl)phosphine; Tris(4-chlorophenyl)phosphine; Di(1-adamantyl)(1-naphthoyl)phosphine; Benzyldiphenylphosphine; 1,1′-Bis(di-t-butylphosphino)ferrocene; (−)-1,2-Bis((2R,5R)-2,5-dimethylphospholano)benzene; (−)-2,3-Bis[(2R,5R)-2,5-dimethylphospholanyl]-1-[3,5-bis(trifluoromethyl)phenyl]-1H-pyrrole-2,5-dione; 1,2-Bis(diphenylphosphino)benzene; 2,2′-Bis(diphenylphosphino)-1,1′-binaphthyl; 2,2′-Bis(diphenylphosphino)-1,1′-biphenyl, 1,4-Bis(diphenylphosphino)butane; 1,2-Bis(diphenylphosphino)ethane; 2-[Bis(diphenylphosphino)methyl]pyridine; 1,5-Bis(diphenylphosphino)pentane; 1,3-Bis(diphenylphosphino)propane; 1,1′-Bis(di-i-propylphosphino)ferrocene; (S)-(−)-5,5′-Bis[di(3,5-xylyl)phosphino]-4,4′-bi-1,3-benzodioxole; tricyclohexylphosphine (referred to herein as PCy3); Tricyclohexylphosphine tetrafluoroborate (referred to herein as PCy3.HBF4); N-[2-(di-1-adamantylphosphino) phenyl]morpholine; 2-(Di-t-butylphosphino)biphenyl; 2-(Di-t-butylphosphino)-3,6-dimethoxy-2′,4′,6′-tri-i-propyl-1,1′-biphenyl; 2-Di-t-butylphosphino-2′-(N,N-dimethylamino)biphenyl; 2-Di-t-butylphosphino-2′-methylbiphenyl; Dicyclohexylphenylphosphine; 2-(Dicyclohexylphosphino)-3,6-dimethoxy-2′,4′,6′-tri-i-propyl-1; 2-(Dicyclohexylphosphino)-2′-(N,N-dimethylamino)biphenyl; 2-Dicyclohexylphosphino-2′,6′-dimethylamino-1,1′-biphenyl; 2-Dicyclohexylphosphino-2′,6′-di-i-propoxy-1,1′-biphenyl; 2-Dicyclohexylphosphino-2′-methylbiphenyl; 2-[2-(Dicyclohexylphosphino)phenyl]-1-methyl-1H-indole; 2-(Dicyclohexylphosphino)-2′,4′,6′-tri-i-propyl-1,1′-biphenyl; [4-(N,N-Dimethylamino)phenyl]di-t-butylphosphine; 9,9-Dimethyl-4,5-bis(diphenylphosphino)xanthene; (R)-(−)-1-[(S)-2-(Diphenylphosphino)ferrocenyl]ethyldicyclohexylphosphine; Tribenzylphosphine; Tri-t-butylphosphine; Tri-n-butylphosphine; and 1,1′-Bis(diphenylphosphino)ferrocene (referred to herein as “DPPF”).

Suitable amine and aminophosphine-based ligands include any combination of monodentate or bidentate alkyl and aromatic amines including, but not limited to, pyridine, 2,2′-Bipyridyl, 4,4′-Dimethyl-2,2′-dipyridyl, 1,10-Phenanthroline, 3,4,7,8-Tetramethyl-1,10-phenanthroline, 4,7-Dimethoxy-1,10-phenanthroline, N,N,N′,N′-Tetramethylethylenediamine, 1,3-Diaminopropane, ammonia, 4-(Aminomethyl)pyridine, (1R,2S,9S)-(+)-11-Methyl-7,11-diazatricyclo[7.3.1.0^{2,7}]tridecane, 2,6-Di-tert-butylpyridine, 2,2′-Bis[(4S)-4-benzyl-2-oxazoline], 2,2-Bis((4S)-(−)-4-isopropyloxazoline)propane, 2,2′-Methylenebis[(4S)-4-phenyl-2-oxazoline], and 4,4′-di-tert-butyl-2,2′bipyridyl. In addition, aminophosphine ligands such as 2-(Diphenylphosphino)ethylamine, 2-(2-(Diphenylphosphino)ethyl)pyridine, (1R,2R)-2-(diphenylphosphino)cyclohexanamine, an aminodextran and 2-(Di-tert-butylphosphino)ethylamine.

Suitable carbene ligands include N-heterocyclic carbene (NHC) based ligands, including, but not limited to, 1,3-Bis(2,4,6-trimethylphenyl)imidazolinium chloride, 1,3-Bis(2,6-diisopropylphenyl)imidazolium chloride, 1,3-Bis-(2,6-diisopropylphenyl) imidazolinium chloride, 1,3-Diisopropylimidazolium chloride, and 1,3-Dicyclohexylbenzimidazolium chloride.

The base used in the reaction mixture is selected to be compatible with the catalyst, the amine and the fluorosulfonate. Suitable bases include, but are not limited to, carbonate salts, phosphate salts, acetate salts and carboxylic acid salts. Inorganic bases are suitable in the reaction mixture. As used herein, “inorganic base” refers to non-organic bases, for example, carbonate salts, phosphate salts, and acetate salts.

Examples of carbonate salts include, but are not limited to, lithium carbonate, sodium carbonate, potassium carbonate, rubidium carbonate, cesium carbonate, ammonium carbonate, substituted ammonium carbonates, and the corresponding hydrogen carbonate salts. Examples of phosphate salts include, but are not limited to, lithium phosphate, sodium phosphate, potassium phosphate, rubidium phosphate, cesium phosphate, ammonium phosphate, substituted ammonium phosphates, and the corresponding hydrogen phosphate salts. Examples of acetate salts include, but are not limited to, lithium acetate, sodium acetate, potassium acetate, rubidium acetate, cesium acetate, ammonium acetate, and substituted ammonium acetates.

Other bases include, but are not limited to, salts of formate, fluoroacetate, and propionate anions with lithium, sodium, potassium, rubidium, cesium, ammonium, and substituted ammonium cations; metal hydroxides, such as lithium hydroxide, sodium hydroxide, potassium hydroxide, metal dihydroxides such as magnesium dihydroxide, calcium dihydroxide, strontium dihydroxide, and barium dihydroxide; metal trihydroxides such as aluminum trihydroxide, gallium trihydroxide, indium trihydroxide, thallium trihydroxide; non nucleophilic organic amines such as triethylamine, N,N-diisopropylethylamine, 1,4-diazabicyclo[2.2.2]octane (DABCO), 1,5-Diazabicyclo[4.3.0]non-5-ene (DBN), 1,8-Diazabicyclo[5.4.0]undec-7-ene (DBU); bis(silyl)amide salts such as the lithium, sodium, and potassium salts of bis(trimethylsilyl)amide; alkoxide salts such as the lithium, sodium, and potassium salts of t butoxide; and 1,8-bis(dimethylamino) naphthalene; metal fluorides, such as sodium fluoride, potassium fluoride, cesium fluoride, silver fluoride, tetra butyl ammonium fluoride, ammonium fluoride, triethyl ammonium fluoride; metal hydrides, such as lithium hydride, sodium hydride and potassium hydride.

Examples of amine bases, such as alkylamines and heteroarenes include, but are not limited to, triethylamine, pyridine, morpholine, 2,6-lutidine, triethylamine, N,N-Dicyclohexylmethylamine, and diisopropylamine.

In one instance, the base is used in the presence of a phase-transfer catalyst. In another instance, the base is used in the presence of water. In yet another instance, the base is used in the presence of an organic solvent. In still another instance, the base is used in the presence of one or more of a phase-transfer catalyst, water or an organic solvent.

Preferably, at least one equivalent of base is present for each equivalent of fluorosulfonate. In some embodiments, no more than 10 equivalents of base are present for each equivalent of fluorosulfonate. In some embodiments, at least 2 equivalents of base are present for each equivalent of fluorosulfonate. In some embodiments, no more than 6 equivalents of base are present for each equivalent of fluorosulfonate.

The solvent in the reaction mixture is selected such that it is suitable for use with the reactants, the catalyst, the ligand and the base. For example, suitable solvents include toluene, xylenes (ortho-xylene, meta-xylene, para-xylene or mixtures thereof), benzene, water, methanol, ethanol, 1-propanol, 2-propanol, n-butanol, 2-butanol, pentanol, hexanol, tert-butyl alcohol, tert-amyl alcohol, ethylene glycol, 1,2-propanedioal, 1,3-propanediol, glycerol, N-methyl-2-pyrrolidone, acetonitrile, N,N-dimethylformamide, methyl acetate, ethyl acetate, propyl acetate, isopropyl acetate, triacetin, acetone, methyl ethyl ketone, and ethereal solvents, such as 1,4-dioxane, tetrahydrofuran, 2-methyltetrahydrofuran, diethylether, cyclopenyl methyl ether, 2-butyl ethyl ether, dimethoxyethane, polyethyleneglycol. In one instance, the solvent includes any combination of the solvents described herein, in, or in the absence of, a surfactant. In one instance, the sulfuryl fluoride is used neat at a sufficiently low temperature that the sulfuryl fluoride is in a liquid. In one instance, water is included in the reaction mixture.

One benefit of using fluorosulfonates as compared to triflates, is that the reaction can be carried out without a subsequent separation step, or with a simple separation step. In couplings involving triflates, a dedicated purification step is required to remove byproducts since the products and the byproducts typically occupy the same phase. In the reaction schemes described herein, the byproducts are either in the gas phase, and will bubble out spontaneously or with a simple degassing step, or will partition into the aqueous phase, which is easily separable. As such, the reaction scheme described herein provides additional benefits as compared to C—N couplings involving triflates.

In one instance, the reaction described herein is completed as a one-pot reaction as shown in Equation 1. In a first step, an compound having an alcohol substituent is added to a reaction mixture in the presence of sulfuryl fluoride and a base. The base may be any of the bases described herein, including, without limitation, amine bases and inorganic bases. This first step couples the fluorosulfonate substituent to the oxygen of the hydroxyl group. To the reaction mixture formed during this first step is added a second compound comprising an amine and a catalyst. The catalyst may be a suitable group 10 catalyst, including, without limitation, platinum, palladium and nickel catalysts. The product of this second step is a compound formed by coupling the first compound and the second compound.

Some embodiments of the invention will now be described in detail in the following Examples. Unless stated otherwise, reported yields are ±5%.

### Example 1

In this Example, para-tolylsulfurofluoridate is reacted with diphenylamine as shown in Equation 5.

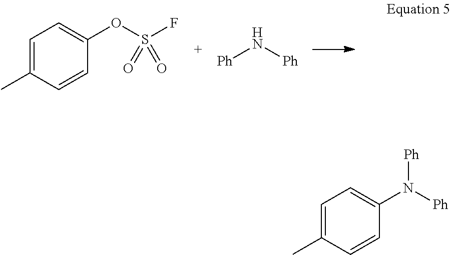 [SMILES: Cc1ccc(OS(=O)(=O)F)cc1 ; c1ccc(Nc2ccccc2)cc1 ; Cc1ccc(N(c2ccccc2)c2ccccc2)cc1]

The present Example is performed in a nitrogen-purged glovebox. A 40 mL glass vial is provided as a reaction vessel. 15 mg of Bis(2-methylallyl)palladium chloride dimer (Strem) is combined with 51 mg of (R)-(−)-1-[(S)-2-(Dicyclohexylphosphino) ferrocenyl]ethyldi-t-butylphosphine (Strem), 1.0 grams of cesium carbonate and 0.31 grams diphenylamine. This mixture is suspended in 6 mL dioxane. p-Tolyl sulfurofluoridate (0.22 mL) is added to the reaction vessel. The reaction vessel is capped and the mixture is stirred with a PTFE-coated stir bar. The reaction vessel is placed in an aluminum heating block at 80° C. and stirred for 60 hours. The mixture is allowed to cool to room temperature and is then diluted with ethyl acetate and then is rinsed with water. The organic layer is isolated and dried over Na₂SO₄. The organic layer is next filtered and the solvent is removed in vacuum. The remaining organic layer is purified by flash chromatography on silica with hexanes and ethyl acetate as the eluent. The solvent is removed in vacuum. The yield of 4-methyl-N,N-diphenylaniline is calculated as 24% (93 mg of product).

### Example 2

In this Example, p-tolyl sulfofluoridate is reacted with benzylamine as shown in Equation 6.

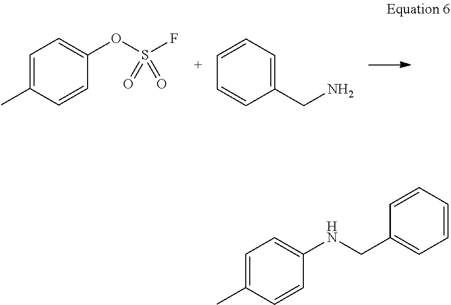

The present Example is performed in a nitrogen-purged glovebox. A 40 mL glass vial is provided as a reaction vessel. 11 mg of cyclopentadienyl[(1,2,3-n)-1-phenyl-2-propenyl]palladium(II) (referred to herein as CpPd (cinnamyl)) (Strem) is combined with 37 mg of 2-(Di-t-butylphosphino)-3,6-dimethoxy-2′,4′,6′-tri-i-propyl-1,1′-biphenyl (Strem), 1.0 grams of cesium carbonate and 0.2 mL benzylamine. This mixture is suspended in 6 mL t-butanol. p-Tolyl sulfurofluoridate (0.22 mL) is added to the reaction vessel. The reaction vessel is capped and the mixture is stirred with a PTFE-coated stir bar. The reaction vessel is placed in an aluminum heating block at 80° C. and stirred for 60 hours. The mixture is allowed to cool to room temperature and is then diluted with ethyl acetate and then is rinsed with water. The organic layer is isolated and dried over Na₂SO₄. The organic layer is next filtered and the solvent is removed in vacuum. The remaining organic layer is purified by flash chromatography on silica with hexanes and ethyl acetate as the eluent. The solvent is removed in vacuum. The yield of N-benzyl-4-methylaniline is calculated as 73% (0.22 grams of product).

### Example 3

In this Example, an aryl fluorosulfonate is reacted with aniline as shown in Equation 7:

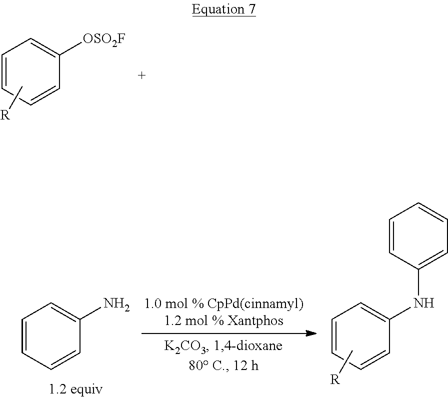

The present Example is performed in a nitrogen-filled glovebox. To each of 12 30 mL vials are added one of the aryl fluorosulfonates selected from Table 1 where R in Equation 7 represents one or more groups joined to the aryl ring as shown (2.50 mmol), Xantphos (0.017 g; 0.03 mmol); potassium carbonate (0.691 g; 5.00 mmol), and 1,4-dioxane (5 mL). To each stirring mixture is added aniline (274 uL) and CpPd (cinnamyl) (0.007 g in 100 uL 1,4-dioxane). Each reaction mixture is heated at 80° C. for 12 to 24 hours. The reaction mixture is cooled to room temperature and adsorbed onto silica gel. The product is purified by flash chromatography (ISCO, manufactured by Teledyne) and the volatiles are removed by vacuum to reveal the desired product having yields recorded in Table 1.

TABLE 1

| | | | | Isolated | |
|---|---|---|---|---|---|
| | | | Yield, | yield, | Time |
| Vial | Fluorosulfonate | Product | mg | % | (h) |
| 1 | 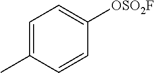 | 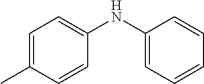 | 415 | 91% | 12 |
| 2 | 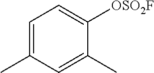 | 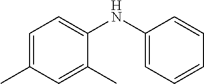 | 478 | 95% | 18 |
| 3 | 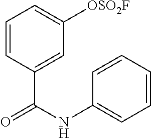 | 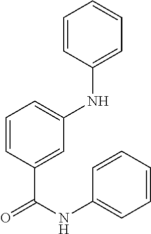 [SMILES: O=C(Nc1ccccc1)c1cccc(Nc2ccccc2)c1] | 645 | 89% | 12 |
| 4 | 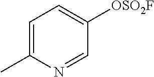 | 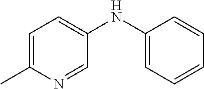 | 440 | 95% | 12 |
| 5 | 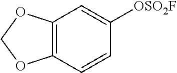 | 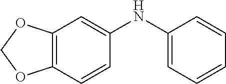 [SMILES: c1ccc(Nc2ccc3c(c2)OCO3)cc1] | 490 | 92% | 18 |
| 6 | 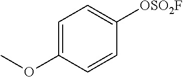 | 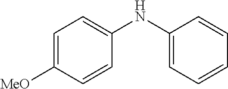 [SMILES: COc1ccc(Nc2ccccc2)cc1] | 331 | 66% | 24 |
| 7 | 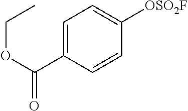 | 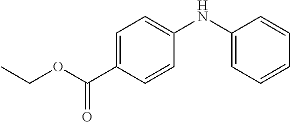 [SMILES: CCOC(=O)c1ccc(Nc2ccccc2)cc1] | 605 | 98% | 12 |
| 8 | 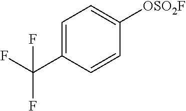 | 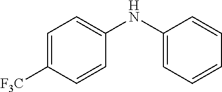 [SMILES: FC(F)(F)c1ccc(Nc2ccccc2)cc1] | 570 | 96% | 12 |
| 9 | 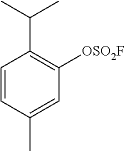 | 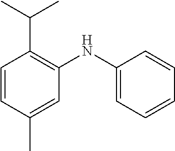 [SMILES: Cc1ccc(C(C)C)c(Nc2ccccc2)c1] | 565 | 97% | 18 |
| 10 | 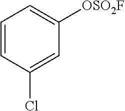 | 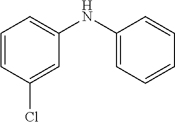 [SMILES: Clc1cccc(Nc2ccccc2)c1] | 487 | 95% | 12 |
| 11 | 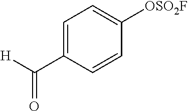 | 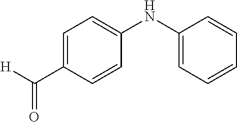 [SMILES: O=Cc1ccc(Nc2ccccc2)cc1] | 466 | 95% | 12 |
| 12 | 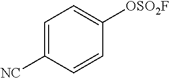 | 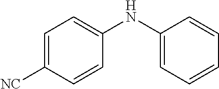 [SMILES: N#Cc1ccc(Nc2ccccc2)cc1] | 500 | 99% | 12 |

### Example 4

In this Example, an aryl fluorosulfonate is reacted with aniline as shown in Equation 8:

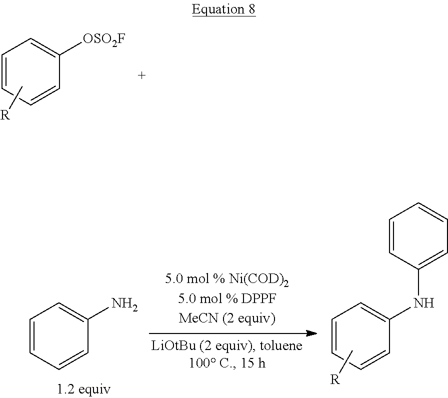

In a nitrogen-filled glovebox, the following reagents are added into each of 13 30 mL scintillation vials: lithium tert-butoxide (0.400 g; 5.00 mmol), Ni(COD)2 (0.034 g; 0.13 mmol), DPPF (0.069 g; 0.13 mmol); an aryl fluorosulfonate selected from Table 2 where R is a substituent joined to the aryl ring as shown (2.50 mmol), toluene (8 mL); aniline (273 μL; 3.00 mmol); and acetonitrile (261 μL). The vials are capped and heated to 100° C. for 15 hours. The vials are cooled to room temperature and GCMS analysis confirmed the presence of desired product in each vial. The reaction mixtures are individually adsorbed onto silica gel and purified by flash. Isolated yields are provided in Table 2.

TABLE 2

| | | | | Isolated |
|---|---|---|---|---|
| | | | Yield, | yield, |
| Vial | Fluorosulfonate | Product | mg | % |
| 1 | 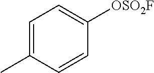 | 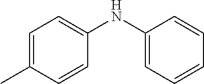 | 297 | 65% |
| 2 | 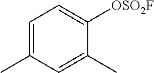 | 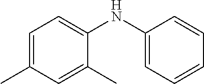 | 216 | 43% |
| 3 | 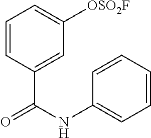 | 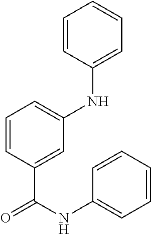 [SMILES: O=C(Nc1ccccc1)c1cccc(Nc2ccccc2)c1] | 120 | 33% |
| 4 | 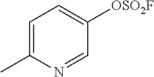 | 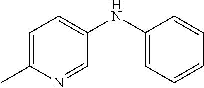 | 157 | 34% |
| 5 | 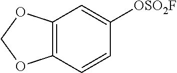 | 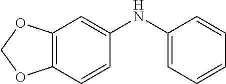 [SMILES: c1ccc(Nc2ccc3c(c2)OCO3)cc1] | 236 | 44% |
| 7 | 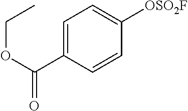 | 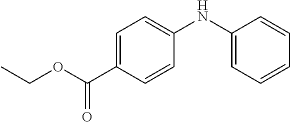 [SMILES: CCOC(=O)c1ccc(Nc2ccccc2)cc1] | 504 | 81% |
| 8 | 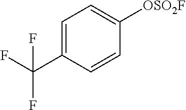 | 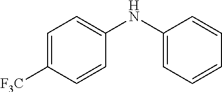 [SMILES: FC(F)(F)c1ccc(Nc2ccccc2)cc1] | 499 | 84% |
| 9 | 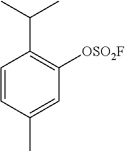 | 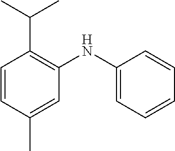 [SMILES: Cc1ccc(C(C)C)c(Nc2ccccc2)c1] | 481 | 83% |
| 10 | 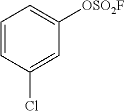 | 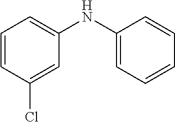 [SMILES: Clc1cccc(Nc2ccccc2)c1] | 436 | 85% |
| 11 | 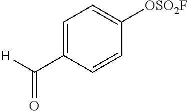 | 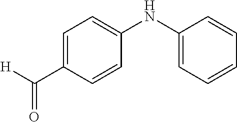 [SMILES: O=Cc1ccc(Nc2ccccc2)cc1] | 284 | 58% |
| 12 | 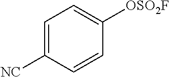 | 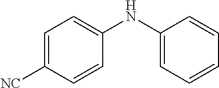 [SMILES: N#Cc1ccc(Nc2ccccc2)cc1] | 398 | 79% |

### Example 5

In this example, a one-pot synthesis is provided to couple ethyl-4-hydroxybenzoate to aniline as shown in Equation 9.

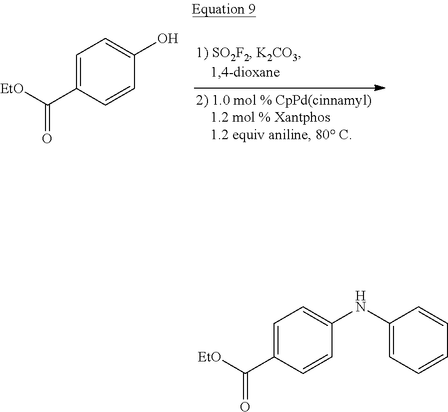 [SMILES: CCOC(=O)c1ccc(O)cc1 ; CCOC(=O)c1ccc(Nc2ccccc2)cc1]

To a 250 mL three-necked round bottomed flask equipped with a thermocouple, condenser vented to a knock-out pot and caustic scrubber, and sulfuryl fluoride gas line is added ethyl-4-hydroxybenzoate (5.0 g; 0.030 mol) and potassium carbonate (12.5 g; 0.090 mol). To the mixture is added 60 mL of 1,4-dioxane. The reaction mixture is stirred at room temperature for 24 hours under a slow bubbling of sulfuryl fluoride to insure an atmosphere of sulfuryl fluoride over the reaction. GCMS analysis revealed conversion of the starting alcohol to the fluorosulfonate is greater than 90%. The sulfuryl fluoride addition line is replaced with a rubber septum, Xantphos (0.209; 0.36 mmol) is added to the reaction mixture, and the reaction mixture is degassed with nitrogen for 20 mins via a needle through the septum. Following completion of scrubbing, the knockout pot is disconnected from the condenser and a nitrogen bubbler is added to the top of the condenser. To the reaction mixture is added aniline (3.3 mL; 0.036 mol) and CpPd(cinnamyl) (0.087 g; 0.30 mmol in 1 mL 1,4-dioxane) via syringe through the septa. The reaction mixture is warmed to 80° C. for 5 hours. GCMS analysis shows complete conversion of the fluorosulfonate to the desired aminated product. The reaction mixture is cooled to room temperature and filtered through a disposable fritted filter. The reaction mixture is adsorbed onto silica gel and purified by flash chromatography. Ethyl-4-(phenylamino)benzoate is collected as an off-white solid. (5.92 g; 82% yield).

### Example 6

In this Example, an aryl fluorosulfonate is reacted with morpholine as shown in Equation 10:

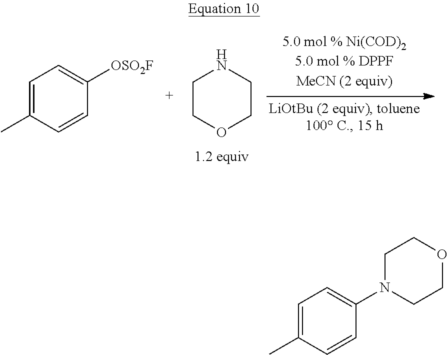

In an N2 filled glovebox, to a 30 mL scintillation vial is added: lithium tert-butoxide (0.400 g; 5.00 mmol), Ni(COD)2 (0.034 g; 0.13 mmol), DPPF (0.069 g; 0.13 mmol); p-tolyl sulfurofluoridate (0.476 g; 2.50 mmol), toluene (8 mL); morpholine (260 uL; 3.00 mmol); and acetonitrile (261 mL). The vial is capped and heated to 100° C. for 15 hours. The reaction is cooled to room temperature and GCMS analysis confirmed the presence of the desired product. The reaction mixture is adsorbed onto silica gel and purified by flash chromatography. 4-(p-tolyl)morpholine is collected as a light brown solid (0.145 g; 33% yield).

What is claimed is:

1. A method of coupling a first compound to a second compound, the method comprising:
providing the first compound having a fluorosulfonate substituent, the first compound comprising an aryl or a heteroaryl group;
providing the second compound comprising an amine; and
reacting the first compound and the second compound in a reaction mixture, the reaction mixture including a catalyst having at least one group 10 atom, the reaction mixture under conditions effective to couple the first compound to the second compound.

2. The method of claim 1, wherein the reaction mixture further includes a ligand.

3. The method of claim 2 wherein the catalyst is generated in-situ from a palladium precatalyst.

4. The method of claim 3 wherein the catalyst is generated in-situ from a palladium precatalyst, the palladium precatalyst is selected from the group consisting of: Palladium(II) acetate, Palladium(II) chloride, Dichlorobis(acetonitrile)palladium(II), Dichlorobis(benzonitrile)palladium(II), Allylpalladium chloride dimer, Palladium(II) acetylacetonate, Palladium(II) bromide, Bis(dibenzylideneacetone)palladium(0), Bis(2-methylallyl)palladium chloride dimer, Crotylpalladium chloride dimer, Dichloro(1,5-cyclooctadiene)palladium(II), Dichloro(norbornadiene)palladium(II), Palladium(II) trifluoroacetate, Palladium(II) benzoate, Palladium(II) trimethylacetate, Palladium(II) oxide, Palladium(II) cyanide, Tris(dibenzylideneacetone)dipalladium(0), Palladium(II) hexafluoroacetylacetonate, cis-Dichloro(N,N,N′,N′-tetramethylethylenediamine)palladium(II), and Cyclopentadienyl[(1,2,3-n)-1-phenyl-2-propenyl]palladium(II), [1,3-Bis(2,6-Diisopropylphenyl)imidazol-2-ylidene](3-chloropyridyl)palladium(II) dichloride, and (1,3-Bis(2,6-diisopropylphenyl)imidazolidene) (3-chloropyridyl) palladium(II) dichloride, and a mixture of two or more thereof.

5. The method of claim 4 wherein the ligand is a phosphine ligand or a carbene ligand.

6. The method of claim 5 wherein the ligand is an amine-based ligand, an aminophosphine-based ligand, an N-heterocyclic carbene-based ligand, a monodentate or bidentate alkyl amine, or a monodentate or bidentate aromatic amine.

7. The method of claim 6 wherein the reaction mixture includes a base.

8. The method of claim 7, wherein the base is a carbonate salt, a phosphate salt, an acetate salt, an alkoxide salt or a carboxylic acid salt.

9. The method of claim 8, wherein the base is selected from the group consisting of lithium carbonate, sodium carbonate, potassium carbonate, rubidium carbonate, cesium carbonate, ammonium carbonate, substituted ammonium carbonates, hydrogen carbonates, lithium phosphate, sodium phosphate, potassium phosphate, rubidium phosphate, cesium phosphate, ammonium phosphate, substituted ammonium phosphates, hydrogen phosphates, lithium acetate, sodium acetate, potassium acetate, rubidium acetate, cesium acetate, ammonium acetate, substituted ammonium acetates, formate salts, fluoroacetate salts, propionate anions with lithium, sodium, potassium, rubidium, cesium, ammonium, and substituted ammonium cations, lithium hydroxide, sodium hydroxide, potassium hydroxide, magnesium dihydroxide, calcium dihydroxide, strontium dihydroxide, and barium dihydroxide, aluminum trihydroxide, gallium trihydroxide, indium trihydroxide, thallium trihydroxide, triethylamine, N,N-diisopropylethylamine, 1,4-diazabicyclo[2.2.2]octane, 1,5-Diazabicyclo[4.3.0]non-5-ene, 1,8-Diazabicyclo[5.4.0]undec-7-ene, lithium, sodium, and potassium salts of bis(trimethylsilyl)amide, lithium, sodium, and potassium salts of t butoxide, 1,8-bis(dimethylamino)naphthalene, pyridine, morpholine, 2,6-lutidine, triethylamine, N,N-Dicyclohexylmethylamine, diisopropylamine, sodium fluoride, potassium fluoride, cesium fluoride, silver fluoride, tetra butyl ammonium fluoride, ammonium fluoride, triethyl ammonium fluoride and a mixture of two or more thereof.

10. The method of claim 9, wherein the reaction mixture includes a solvent.

11. The method of claim 10, wherein the solvent is selected from the group consisting of toluene, xylene, benzene, chlorobenzene, water, methanol, ethanol, 1-propanol, 2-propanol, n-butanol, 2-butanol, pentanol, hexanol, tert-butyl alcohol, tert-amyl alcohol, ethylene glycol, 1,2-propanedioal, 1,3-propanediol, glycerol, N-methyl-2-pyrrolidone, acetonitrile, N,N-dimethylformamide, methyl acetate, ethyl acetate, propyl acetate, isopropyl acetate, triacetin, acetone, methyl ethyl ketone, and ethereal solvents, such as 1,4-dioxane, tetrahydrofuran, 2-methyltetrahydrofuran, diethylether, cyclopenyl methyl ether, 2-butyl ethyl ether, dimethoxyethane, polyethyleneglycol, dimethylacetamide (DMA), dimethylsulfoxide (DMSO), and 1,2-dichloroethane (DCE).

12. The method of claim 11, wherein the reaction mixture includes water.

13. A method for coupling a first compound A¹ a second compound A², as illustrated in Equation 1, comprising: 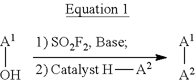
providing the first compound A¹ having a hydroxyl substituent, sulfuryl fluoride and a base to a reaction mixture, the first compound A¹ comprising an aryl group or a heteroaryl group;
providing a catalyst comprising a group 10 atom and the second compound A² to the reaction mixture, the second compound A² as defined in Equation 4: 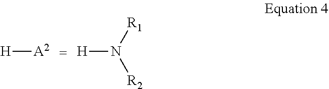
the second compound A² comprising an amine wherein each of R¹ and R² are independently Hydrogen, an aryl group, a heteroaryl group, an alkyl group, a cycloalkyl group, a nitro group, a halide, a nitrogen, a cyano group, a carboxyester group, an acetoxy group, a substituted alkyl, aryl, heteroaryl or cycloalkyl group, or R¹ and R² are constituent parts of a ring system;
reacting the first compound and the second compound under conditions effective to couple the first compound to the second compound.

14. The method of claim 13, wherein the reaction mixture further includes a ligand, and a base.

15. The method of claim 14 wherein the catalyst is generated in-situ from a palladium precatalyst.

16. The method of claim 15 wherein the ligand is a phosphine ligand or a carbene ligand.

17. The method of claim 16 wherein the ligand is an amine-based ligand, an aminophosphine-based ligand or an N-heterocyclic carbene-based ligand.

18. The method of claim 17 wherein the ligand is a monodentate or bidentate alkyl amine or a monodentate or bidentate aromatic amine.

19. The method of claim 18, wherein the base is a carbonate salt, a phosphate salt, an acetate salt or a carboxylic acid salt.

20. A method comprising:
providing to a first mixture, said first mixture comprising ethyl-4-hydroxybenzoate, sulfuryl fluoride, a base and a solvent,
wherein said base is selected from the group consisting of lithium carbonate, lithium hydrogen carbonate, sodium carbonate, sodium hydrogen carbonate, potassium carbonate, potassium hydrogen carbonate, rubidium carbonate, rubidium hydrogen carbonate, cesium carbonate, cesium hydrogen carbonate, ammonium carbonate, and ammonium hydrogen carbonate, and
wherein said solvent is selected from the group consisting of 1,4-dioxane, tetrahydrofuran, 2-methyltetrahydrofuran, diethylether, cyclopenyl methyl ether, 2-butyl ethyl ether, dimethoxyethane, polyethyleneglycol, and mixtures thereof;
reacting said first mixture to provide a second mixture comprising ethyl 4-((fluorosulfonyl)oxy)benzoate;
providing a phosphine ligand to said second mixture, wherein said phosphine ligand is selected from the group consisting of triphenylphosphine, tri(o-tolyl)phosphine, tris(4-methoxyphenyl)phosphine, tris(pentafluorophenyl)phosphine, 9,9-dimethyl-4,5-bis(diphenylphosphino)xanthene, (R)-(−)-1-[(S)-2-(diphenylphosphino)ferrocenyl]ethyldicyclohexylphosphine, and 1,1′-bis(diisopropylphosphino)ferrocene;
removing sulfuryl fluoride if present, from said second mixture;
providing aniline and a precatalyst to said second mixture, wherein said precatalyst is selected from the group consisting of palladium(II) acetate, palladium(II) chloride, dichlorobis(acetonitrile)palladium(II), dichlorobis(benzonitrile)palladium(II), palladium(II) acetylacetonate, dichloro(1,5-cyclooctadiene)palladium(II), dichloro(norbomadiene)palladium(II), tris(dibenzylideneacetone)dipalladium(0), palladium(II) hexafluoroacetylacetonate, cis-dichloro(N,N,N′,N′-tetramethylethylenediamine)palladium(II), and cyclopentadienyl[(1,2,3-n)-1-phenyl-2-propenyl]palladium(II); and
reacting said second mixture comprising ethyl 4-((fluorosulfonyl)oxy)benzoate, aniline, said base, said solvent, said ligand, and said precatalyst under conditions effective to couple ethyl 4-((fluorosulfonyl)oxy)benzoate to aniline to provide ethyl 4-(phenylamino)benzoate.
